(19) United States
(12) Patent Application Publication
(10) Pub. No.: US 2004/0086951 A1
(43) Pub. Date: May 6, 2004

(54) Method for detecting in/vitro food antigen intolerance

(76) Inventors: Alexandr Ivanovich Archakov, Moscow (RU); Natalia Victorovna Semenova, Moscow (RU); Irina Borisovna Makarova, Moscow (RU)

Correspondence Address: Thomas M Galgano, Galgano & Burke, Suite 135, 300 Rabro Drive, Hauppauge, NY, 11788, US

(21) Appl. No.: 10/468,332

(22) PCT Filed: Aug. 23, 2002
(86) PCT No.: PCT/RU02/00396

(30) Foreign Application Priority Data
Aug. 24, 2001 (RU) 2001123575

(51) Int. Cl. G01N 33/567

(52) U.S. Cl. 435/7.21

(57) ABSTRACT

The invention relates to medicine, more specifically, to immunoalergy and is for detecting intolerance of alimentary antigens.

The invention has for its technical aim to accelerate and simplify a method for detecting intolerance of alimentary antigens in adults and children.

The method resides incubating an alimentary antigen together with the cells of patient's heparinized blood.

The method resides in incubating an alimentary antigen together with the cells of patient's heparinized blood. Studies are conducted with venous blood, incubation is performed with the antigen diluted to a concentration of 1:5000 PNU), metabolic activation of granulocytes is assessed against their post-incubation percentage, and when said percentage is found to have been raised as compared to the norm, intolerance of the alimentary antigen involved is revealed.

### TECHNICAL FIELD

This invention relates to medicine, more particularly to immunoallergology and is aimed at detecting intolerance of alimentary (ingestant) antigens.

### BACKGROUND ART

At present the most widespread methods for in-vitro detection of personal intolerance of alimentary (ingestant) antigens are those involving use of immunoenzymometric assay (cf. Gevazieva V. B. et al., “Use of solid-phase immunoenzymometric assay for detecting allergen-specific Ig E antibodies”, JMEI, 1987 #9, pp. 33-35 (in Russian) or of fluorescent probes (cf. Kirillov M. A., “Diagnosis of specific sensibilization and functional state of leukocyte membranes in allergic diseases of children, using fluorescence probes”, M.Sc. Dissertation, Leningrad, 1991 (in Russian).

However the aforesaid methods are multi-stage and long-continued ones, involve the use of costly test-systems and reagents containing highly potent or toxic substances.

In recent years more acceptable and less toxic for use became such methods for detecting food intolerance that are concerned with studies of blood corpuscles (cf. RF Patents ##2,094,805, 1997 and 2,140,085, 1999).

As the closest works may be regarded those where food antigens were studied in the reaction of inhibition of natural leukocyte migration (cf. a paper by Potemkina A. M. and Gizatullina N. R. “Test for inhibition of natural leukocyte migration in diagnosis of alimentary allergy”, The Kazan medical journal, 1993 #5, pp. 353-355 (in Russian), as well as studies into morphology of eosinophils by a method of blood incubation with an alimentary allergen after a two-hour incubation under physiological conditions (cf. E. S. Nenasheva et al. “Method for diagnosis of allergy by studying morphology of eosinophils”, Clinical laboratory diagnostics, 1995, #2, pp. 29-31 (in Russian).

### Essence of the invention

The present invention has for its technical object to provide an accelerated and simplified method for detecting intolerance of alimentary allergens in adults and children.

Said object is accomplished due to studying changes in metabolic response of blood granulocytes to their in-vitro incubation with alimentary antigens.

### EMBODIMENTS OF THE INVENTION

The present invention is carried out as follows. Venous blood is taken from a patient, then is subjected to heparinization and incubation together with an alimentary antigen for 15-20 min under physiological conditions, said alimentary antigen being used in a concentration of 1000, 5000, and 10000 PNU (protein nitrogen unit) per milliliter. For assessing the results there is determined percentage of granulocytes activated by alimentary antigens with the aid of a flow cytofluorimeter and using chemiluminescence technique. Functional activity of cells is determined in all patients using bacterial (E. coli) activator tests, and metabolic potential, using PMA (phorbol myristate acetate) tests. Metabolic reserve and phagocytic activity are retained in all the patients who have been examined, whereby the results of the blood tests of said patients are used for elaborating a blood test for alimentary antigen intolerance.

Intolerance of the following alimentary antigens have been studied: whole eggs, milk, tangerine, cod, pork, beef, hen's meat, wheat, and rice.

Estimation of the level of class Ig E specific antibodies in blood serum using a multiple allergosorbent chemiluminescence test and leukocyte agglomeration reaction with the same alimentary antigens are made use of as control methods.

The results of the aforesaid studies are assessed against percentage of alimentary antigen-activated granulocytes and by a coefficient of alimentary antigen activation intensity of granulocytes. A total of 20 patients aged from two to 55 years have been tested. No intolerance of the alimentary antigens is detected in 15 patients who has been subjected to control tests (for specific Ig E and leukocyte agglomeration reaction. Five patients exhibit high titers of specific antibodies to alimentary antigens.

Statistical treatment has revealed as follows:

1. Percentage of activated granulocytes in a group of patients exhibiting good alimentary antigen tolerance in tests with a 1:10000 concentration is M±m=3.403±0.590%, (n=59).

The coefficient of alimentary antigen activation intensity of granulocytes M±m=1.015±0.077%, (n=62).

Percentage of activated granulocytes in tests with a 1:5000 concentration is M±m=5.632±0.760%, (n=18).

The coefficient of alimentary antigen activation intensity of granulocytes M±m=1.134±0.128%, (n=18).

2. Percentage of activated granulocytes in a group of patients exhibiting alimentary antigen intolerance in tests with a 1:10000 concentration is M±m=5.017±1.179%, (n=18).

The coefficient of alimentary antigen activation intensity of granulocytes M±m=1.53±0.109%, (n=18).

Percentage of activated granulocytes in tests with a 1:1000 concentration is M±m=13.867±2.735%, (n=27).

The coefficient of alimentary antigen activation intensity of granulocytes M±m=1.310±0.091%, (n=45).

Percentage of activated granulocytes in tests with a 1:5000 concentration is M±m=11.06±1.0%, (n=45).

Percentage of activated granulocytes in tests with a 1:1000 concentration is M±m=13.867±2.735%, (n=27).

The coefficient of alimentary antigen activation intensity of granulocytes M±m=1.251±0.101%, (n=27).

The coefficient of alimentary antigen activation intensity of granulocytes is calculated as the ratio of the granulocyte activation intensity in an alimentary antigen test to a normal test (Table 1).

### Industrial Applicability

Sensitivity of the herein-proposed test for alimentary antigen intolerance is 70%.

Specificity of the test is 93.55%.

Predictive value of a positive test result is 95.12%.

Predictive value of a negative test result is 82.9%.

TABLE 1

| | |
|---|---|
| Percentage of alimentary | Coefficient of alimentary |
| antigen activation | antigen activation |
| intensity of granulocytes | intensity of granulocytes |

| Norm | Intolerance | Norm | Intolerance |
|---|---|---|---|
| Alimentary antigen | | Alimentary antigen | |
| concentration 1:10000 PNU | | concentration 1:10000 PNU | |
| 3.403 ± 0.590 | 5.017 ± 1.179 | 1.015 ± 0.077 | 1.53 ± 0.109 |
| Alimentary antigen | | Alimentary antigen | |
| concentration 1:5000 PNU | | concentration 1:5000 PNU | |
| 5.632 ± 0.760 | 11.06 ± 1.00 | 1.134 ± 0.128 | 1.319 ± 0.91 |

1. A method for in-vitro detection of intolerance of alimentary antigen, comprising its incubation together with the cells of patient's heparinized blood, CHARACTERIZED in that studies are carried out in patient's venous blood, incubation is effected together with the antigen diluted to a concentration of 1:5000 PNU (protein nitrogen unit), metabolic activation of granulocytes is assessed against their post-incubation percentage, and when said percentage is found to have been raised as compared to the norm, intolerance of the alimentary antigen involved is revealed.

2. A method as claimed in claim 1, CHARACTERIZED in that incubation is carried out under physiological conditions for 15-20 min.

3. A method as claimed in claim 1, CHARACTERIZED in that metabolic activation of granulocytes is assessed with the aid of a flow cytofluorimeter or recorded in a chemiluminometer.
